(19) United States
(12) Patent Application Publication
(10) Pub. No.: US 2009/0048336 A1
(43) Pub. Date: Feb. 19, 2009

(54) ESCITALOPRAM OXALATE POWDERS

(76) Inventors: Naveen Kumar Kolla, Hyderabad (IN); Ravi Ram Elati, Hyderabad (IN); Srinivas Gangula, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/193,201

(22) Filed: Aug. 18, 2008

Related U.S. Application Data
(60) Provisional application No. 61/039,159, filed on Mar. 25, 2008.

(30) Foreign Application Priority Data
Aug. 17, 2007 (IN) 1835/CHE/2007

(51) Int. Cl. A61K 31/343; C07D 307/87; A61P 25/24

(52) U.S. Cl. 514/469; 549/467

(57) ABSTRACT

Escitalopram oxalate powders having definite particle size distribution parameters, processes for preparing the powders, and solid pharmaceutical formulations containing the powders.

### INTRODUCTION

The present application relates to escitalopram oxalate powders having definite particle size distribution parameters and to processes for the preparation thereof. More specifically, the present application relates to escitalopram oxalate wherein ratios between D₅₀ and D₉₅ are greater than 0.42. It also relates to pharmaceutical compositions that include such powders and their use in the therapy of depression.

Escitalopram oxalate, chemically known as S-(+)-1-[3-(dimethyl-amino)propyl]-1-(p-fluorophenyl)-5-phthalancarbonitrile oxalate, is represented structurally by Formula I.

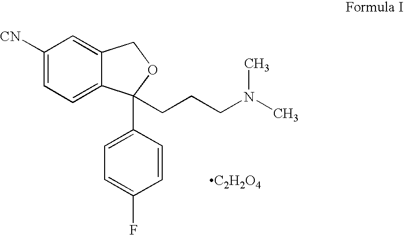

Escitalopram is the S-enantiomer of the racemic bicyclic phthalane derivative citalopram. Escitalopram is a selective serotonin reuptake inhibitor useful in the treatment of depression and is commercially available in products sold as LEXAPRO™.

U.S. Pat. No. 6,916,941 discloses crystalline escitalopram oxalate having median particle sizes in the range of 50-200 μm.

U.S. Patent Application Publication No. 2005/0196453 describes particles of escitalopram oxalate having median particle sizes at least 40 μm, and ratios between median particle size and the particle size at the 95% quantile less than 0.42.

International Application Publication No. WO 2008/046617 discloses escitalopram having small particle sizes and specific ratios of particle size distribution parameters.

Escitalopram is a low-dose active substance. It is desirable to have a narrow particle size distribution in order to obtain constant uniformity of the active substance contents in pharmaceutical dosage forms within the compendial limits.

### SUMMARY

The present application provides escitalopram oxalate powders with definite particle size distribution parameters and processes for the preparation thereof. It also provides pharmaceutical compositions that include such particles, and their uses.

In an embodiment, the present application provides escitalopram oxalate powder having ratios of D₅₀ to D₉₅ greater than 0.42.

In an embodiment, the present application provides escitalopram oxalate powder having ratios of D₅₀ to D₉₅ greater than 0.42 and less than 0.6.

In another embodiment, the present application provides escitalopram oxalate powder having sizes D₅₀ less than about 10 μm, or in the range of about 3 μm to about 7 μm.

An aspect provides processes for manufacturing escitalopram oxalate with defined particle sizes, an embodiment comprising:

1) providing a solution of escitalopram in an organic solvent;

2) reacting with oxalic acid to produce escitalopram oxalate and cause its precipitation as a solid;

3) isolating a solid; and

4) micronising the solid to obtain an escitalopram oxalate powder having defined particle size distribution parameters.

Each process step is contemplated separately and in the context of a multi-step sequence.

The present patent application also provides solid dosage forms that include escitalopram oxalate having particle size distribution parameters described herein.

### DETAILED DESCRIPTION

As set forth herein, an aspect of the invention provides processes for manufacturing escitalopram oxalate powders having defined particle size distribution parameters, an embodiment comprising:

1) providing a solution of escitalopram in an organic solvent;

2) reacting with oxalic acid to produce escitalopram oxalate and cause its precipitation as a solid;

3) isolating the solid; and

4) micronising the solid to obtain escitalopram oxalate having defined particle size parameters.

The initial step involves providing a solution of escitalopram free base in an organic solvent. Useful solvents include those in which the free base is soluble, and in which the oxalate salt has limited solubility. The solution may be obtained by dissolving the free base in a solvent, or the solution may be obtained directly from a manufacturing step in which escitalopram free base is obtained. Non-limiting examples of organic solvents that may be used for dissolution of the free base include: esters, such as ethyl acetate and propyl acetate; C₁-C₅ ketones, such as acetone, ethyl methyl ketone, and butanone; and mixtures thereof in various proportions without limitation.

The dissolution temperatures may range from about 25° C. to about 100° C. The dissolution times may be as long as necessary to complete the dissolution; dissolution times from about 30 minutes to about 10 hours being appropriate in certain instances.

Following solution formation, the escitalopram solution is reacted with oxalic acid to obtain escitalopram oxalate salt. In embodiments, the oxalic acid is used in the form of a solution in a solvent. Examples of solvents that may be used include but are not limited to: esters, such as ethyl acetate and propyl acetate; and C₁-C₅ ketones, for example, acetone, ethyl methyl ketone, and butanone.

Upon addition of oxalic acid, the formation of the oxalate salt occurs. The salt is less soluble in the solvent than is escitalopram, and thus the salt begins to precipitate. To enhance solid formation, the reaction mass may be allowed to stand and/or cooled, as desired for a more complete solid formation. The solid mass of escitalopram oxalate is recovered by suitable techniques, such as, for example, decantation, filtration by gravity or by suction, centrifugation, and the like. The solid may also be dried.

Drying can be carried out in a tray dryer, vacuum oven, air oven, fluidized bed drier, spin flash dryer, flash dryer, and the like. Drying may be carried out at temperatures from about 25° C. to about 75° C., with or without vacuum, and in the presence or absence of an inert atmosphere like nitrogen, argon, neon, and helium. Drying may be carried out for any desired time periods to achieve the desired product purity.

The obtained dry solid may contain small amounts of lumps or agglomerated material. A uniform, free flowing, fine solid may be obtained by sieving, gas jet milling, pulverization and other methods. To afford uniform, free flowing particles, a mesh size used for sieving the escitalopram oxalate may be in the range of from about 20 to about 40 mesh. In a variant, the sieve is 30 mesh. If desired, the obtained particles may be dried again.

The powder obtained from sieving may be micronized to afford a solid with definite particle size distributions. Feed pressure in a microniser that has been used for the example below may range from about 1 to about 4 Kg/cm². In a variant, the feed pressure is about 2 Kg/cm². The chamber pressure may range from about 0.5 to about 2 Kg/cm². In a variant, the chamber pressure is 1.5 Kg/cm². The feed rate may range from about 1 to about 3 Kg/hour. In a variant, the feed rate is about 1 to about 2 Kg/hour. To afford the desired particle sizes, the process may be repeated.

Also provided are escitalopram oxalate powders obtained by the above process having D₅₀ less than about 10 μm. In embodiments, D₅₀ is from about 3 μm to about 7 μm.

Further provided are escitalopram oxalate powders, having particle size distributions wherein ratios of D₅₀ to D₉₅ are greater than 0.42. In embodiments, escitalopram oxalate powders have particle size distributions wherein ratios of D₅₀ to D₉₅ are at least about 0.43. Other embodiments provide escitalopram oxalate powders having particle size distributions wherein ratios of D₅₀ to D₉₅ are at least about 0.44. Further embodiments provide escitalopram oxalate powders having particle size distributions wherein ratios of D₅₀ to D₉₅ are at least about 0.45. In embodiments, escitalopram oxalate powders having the above ratios will also have maximum ratios that are not greater than 0.6.

The term “particle size distribution” as used herein refers to the relative percentages by weight or volume of each of the different size fractions of a particulate matter. The term “median particle size” as used herein refers to the median or 50% quantile of a particle size distribution.

The term D₉₅ as used herein is defined as a size of particles where 95 volume percent of the particles have sizes less than the value given. The term D₅₀ defines a size where 50 volume percent of the particles have sizes less than the value given.

The particle size distributions for the present application can measured using laser light diffraction equipment, such as are sold by Malvern Instruments Ltd., Malvern, Worcestershire, United Kingdom. Other types of equipment are also suitable for particle size distribution determinations.

Also provided area solid dosage forms that include escitalopram oxalate with any of the particle size characteristics described herein. While it is understood that the particle sizes of escitalopram oxalate present in a solid dosage form may or may not be measurable (e.g., after compression to form tablets), the particle size characteristics of the starting powdered escitalopram oxalate are measurable. The pharmaceutical solid oral dosage forms include tablets, capsules, granules etc.

The compositions may further include one or more excipients such as diluents, binders, disintegrants, glidants, lubricants, colorants, solvents, film-forming polymers, plasticizers, opacifiers, anti-adhesives, and polishing agents.

### Diluents:

Various useful diluents include but are not limited to starches, lactose, mannitol, pearlitol SD 200, cellulose derivatives, confectioners' sugar and the like. Different grades of lactose include but are not limited to lactose monohydrate, lactose DT (direct tableting), lactose anhydrous, Flowlac™ (available from Meggle Products), Pharmatose™ (available from DMV) and others. Different grades of starches included but not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different cellulose compounds that can be used include crystalline cellulose and powdered cellulose. Examples of crystalline cellulose products include but are not limited to CEOLUS™ KG801, Avicel™ PH 101, PH102, PH301, PH302 and PH-F20, microcrystalline cellulose 114, and microcrystalline cellulose 112. Other useful diluents include but are not limited to carmellose, sugar alcohols such as mannitol, sorbitol and xylitol, calcium carbonate, magnesium carbonate, dibasic calcium phosphate, and tribasic calcium phosphate.

### Binders:

Various useful binders include but are not limited to hydroxypropylcelluloses (Klucel™ LF), hydroxypropyl methylcelluloses or hypromelloses (Methocel™), polyvinylpyrrolidones or povidones (PVP-K25, PVP-K29, PVP-K30, PVP-K90), Plasdone™ S 630 (copovidone), powdered acacia, gelatin, guar gum, carbomers (e.g. Carbopol™), methylcelluloses, polymethacrylates, and starch.

### Disintegrants:

Various useful disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (FMC-Asahi Chemical Industry Co., Ltd.), crospovidones, and low-substituted hydroxypropylcelluloses. Examples of commercially available crospovidone products include but not limited to crosslinked povidone, Kollidon™ CL [manufactured by BASF (Germany)], Polyplasdone™ XL, XI-10, and INF-10 [manufactured by ISP Inc. (USA)], Examples of low-substituted hydroxypropylcelluloses include but are not limited to low-substituted hydroxypropylcelluloses LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starches.

### Glidants:

Various glidants or antisticking agents include but are not limited to talc, silica derivatives, colloidal silicon dioxide and the like, and mixtures thereof.

### Lubricants:

Various lubricants that can be used include but are not limited to stearic acid and stearic acid derivatives such as magnesium stearate, calcium stearate, zinc stearate, sucrose esters of fatty acid, polyethylene glycol, talc, sodium stearyl fumarate, zinc stearate, castor oils, and waxes.

### Colourants:

Various useful colourants include but are not limited to Food Yellow No. 5, Food Red No. 2, Food Blue No. 2, and the like, food lake colorants, and iron oxides.

### Film-Forming Agents:

Various film-forming agents include but are not limited to: cellulose derivatives such as soluble alkyl- or hydroalkylcellulose derivatives such as methylcelluloses, hydroxymethyl celluloses, hydroxyethyl celluloses, hydroxypropyl celluloses, hydroxymethyethyl celluloses, hydroxypropyl methylcelluloses (HPMC, different grades such as HPMC 6 cps, HPMC 15 cps, and HPMC 50 cps being available), sodium carboxymethyl celluloses, etc., acidic cellulose derivatives such as cellulose acetate phthalates, cellulose acetate trimellitates, methylhydroxypropylcellulose phthalates, polyvinyl acetate phthalates, etc., insoluble cellulose derivatives such as ethylcelluloses and the like; dextrins, starches and starch derivatives, polymers based on carbohydrates and derivatives thereof; natural gums such as gum Arabic, xanthans, alginates; polyacrylic acids; polyvinyl alcohols; polyvinyl acetates; polyvinylpyrrolidones; polymethacrylates and derivatives thereof (Eudragit™); chitosan and derivatives thereof; shellac and derivatives thereof; waxes; and fat substances.

If required, the films may contain additional adjuvants for coating processing such as plasticizers, polishing agents, colorants, pigments, antifoam agents, opacifiers, antisticking agents, and the like.

### Plasticizers:

Various plasticizers include but are not limited to castor oil, diacetylated monoglycerides, dibutyl sebacate, diethyl phthalate, glycerin, polyethylene glycols, propylene glycols, triacetin, and triethyl citrate. Also, mixtures of plasticizers may be utilized. The type of plasticizer depends upon the type of coating agent. A plasticizer is normally present in amounts ranging from about 5% (w/w) to about 30% (w/w), based on a total weight of the film coating.

An opacifier like titanium dioxide may also be present in an amount ranging from about 10% (w/w) to about 20% (w/w), based on the total weight of the coating. When coloured tablets are desired, then the colour is normally applied in the coating. Consequently, colouring agents and pigments may be present in the film coating. Various colouring agents include but not limited to iron oxides, which can be red, yellow, black or blends thereof.

Anti-adhesives are frequently used in film coating processes to avoid sticking effects during film formation and drying. An example of an anti-adhesive for this purpose is talc. The anti-adhesive can be present in the film coating in amounts of about 5% (w/w) to about 15% (w/w), based upon the total weight of the coating.

Suitable polishing agents include polyethylene glycols of differing molecular weights or mixtures thereof, talc, surfactants (e.g., glycerol monostearate and poloxamers), fatty alcohols (e.g., stearyl alcohol, cetyl alcohol, lauryl alcohol and myristyl alcohol) and waxes (e.g., carnauba wax, candelilla wax and white wax). In embodiments, polyethylene glycols having molecular weights of 3,000-20,000 are employed.

As an alternative to the above coating ingredients, pre-formulated coating products such as those sold as OPADRY™ (supplied by Colorcon) may also be used. The dry products require only dispersion in a liquid before use.

### Solvents:

Representative solvents used in the processes of preparation of pharmaceutical compositions of the present invention include, but are not limited to, water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethyl sulphoxide, dimethyl formamide, tetrahydrofuran, and mixtures thereof.

The present application provides solid unit dosage forms prepared using escitalopram oxalate with definite particle size distribution parameters, and are prepared by techniques such as direct compression, dry granulation or wet granulation. Equipment suitable for processing the pharmaceutical compositions of the present invention include mechanical sifters, blenders, roller compactor, compression machine, rotating bowls or coating pans, etc.

The term “wet granulation” as used in this invention refers to a process of adding a liquid to a powder mixture, while mixing until granules are formed.

Having thus described the invention with reference to particular embodiments and illustrative examples, those in the art will appreciate modifications to the invention as described and illustrated that do not depart from the spirit and scope of the invention as disclosed in the specification. The following examples are set forth to further describe certain specific aspects and embodiments of the invention but are not intended to, and should not be construed to limit its scope in any way. The examples do not include detailed descriptions of conventional methods. Such methods are well known to those of ordinary skill in the art and are described in numerous publications.

### EXAMPLE 1

### Preparation of Escitalopram Oxalate

Ethyl acetate (52 L) was charged into a reactor followed by the addition of oxalic acid (2.54 Kg). The mass was heated to about 45° C. for 25 minutes. The obtained solution was filtered through a micro filter to remove undissolved material.

Ethyl acetate (10 L) was charged into another reactor containing escitalopram free base (6.6 Kg) and the mixture was stirred for 15 minutes to form a solution. The oxalic acid solution prepared above was added slowly into the escitalopram solution over a period of 60 minutes at about 25° C. The resultant reaction mixture was heated to 65° C. and stirred for 90 minutes. The reaction mixture was cooled to about 0° C. and stirred for 2 hours. The formed solid was centrifuged and washed with ethyl acetate (7 L). The obtained solid was again charged into a reactor containing ethyl acetate (39 L) and the mass was heated to 70° C. and stirred for 40 minutes. The mixture was cooled to 0° C. and stirred for two hours. The solid was filtered, washed with ethyl acetate (7 L) and dried.

The obtained solid was loaded into a cone vacuum dryer and dried at 55° C. for 2 hours. The dried material was sieved through a 30 mesh sieve to obtain an agglomerate-free uniform particle size distribution. The fine powder was again loaded into a cone vacuum dryer and dried at 55° C. under vacuum for 4 hours to afford the title compound (10.54 Kg).

The obtained solid was micronized in a jet mill to produce desired particle sizes. The feed pressure was adjusted to about 2 Kg/cm²; the chamber pressure was adjusted to about 1.5 Kg/cm² and the feed rate to about 1 to 2 kg/hour. The material fed into the micronizer through a charging hopper. The obtained micronized material was collected into a container.

The resulting escitalopram oxalate batches prepared by the above process had particle size distribution characteristics as shown in Table 1, wherein the parameters were determined using a Malvern instrument.

TABLE 1

| Batch | D₅₀ | D₉₅ | D₅₀/D₉₅ |
|---|---|---|---|
| 1 | 3.95 | 9.20 | 0.4293 |
| 2 | 4.56 | 9.80 | 0.4653 |

### EXAMPLE 2

### Preparation of Pharmaceutical Composition

| Ingredient | mg/Tablet |
|---|---|
| Escitalopram oxalate | 6.388 |
| Fumed silica (Aerosil ™ 200) | 1.25 |
| Lactose monohydrate | 12.41 |
| Povidone K-30 | 0.2 |
| Microcrystalline cellulose (Avicel ™ | 41 |
| PH101) | |
| Croscarmellose sodium | 1.25 |
| Talc | 1.25 |
| Magnesium stearate | 0.75 |
| Aerosil 200 | 0.5 |
| Opadry ™ coating | 1.62 |
| Total (Coated Tablet) | 66.62 |

1) Escitalopram oxalate, Aerosil (first quantity) and lactose monohydrate were sifted through a 40 mesh sieve.
2) The sifted material was loaded into a rapid mixer/granulator (RMG) and mixed for about 15 minutes with impeller running at a fast speed and the chopper off.
3) Povidone K-30 was dissolved in water.
4) Binder solution of 3) was added to the dry mix of 2), while running the RMG impeller at a fast speed and the chopper off.
5) The granules were dried in a fluid bed dryer.
6) The dried granules were sifted through a 30 mesh sieve, and the retained particles were milled through a comminuting mill with a 1 mm screen. Milled particles were passed through a 30 mesh sieve and combined with the first sieved material.
7) Avicel PH101 and croscarmellose sodium were sifted through a 40 mesh sieve.
8) Magnesium stearate, talc and Aerosil (second quantity) were sifted through a 60 mesh sieve.
9) The granules of 6) were blended with the material of 7) in a low shear mixer.
10) The mixture of 9) was blended with the material obtained in 8) in a low shear mixer.
11) The blend of 10) was compressed into tablets at a target weight of 65 mg.
12) Opadry coating was dispersed in water and the tablets were coated to produce a weight gain of 2.5% w/w.

We claim:

1. An escitalopram oxalate powder having ratios of D₅₀ to D₉₅ greater than 0.42.

2. The escitalopram oxalate powder of claim 1, wherein ratios of D₅₀ to D₉₅ are at least about 0.43.

3. The escitalopram oxalate powder of claim 1, wherein ratios of D₅₀ to D₉₅ are at least about 0.44.

4. The escitalopram oxalate powder of claim 1, wherein ratios of D₅₀ to D₉₅ are at least about 0.45.

5. The escitalopram oxalate powder of claim 1, wherein ratios of D₅₀ to D₉₅ are not greater than 0.6.

6. The escitalopram oxalate powder of claim 1, wherein D₅₀ sizes are less than about 10 μm.

7. The escitalopram oxalate powder of claim 1, wherein D₅₀ sizes are about 3 μm to about 7 μm.

8. A process for preparing an escitalopram oxalate powder, comprising:
(a) providing a solution of escitalopram in an organic solvent;
(b) adding oxalic acid to produce escitalopram oxalate and causing precipitation of a solid;
(c) isolating a solid; and
(d) micronising the solid to form a powder having ratios of D₅₀ to D₉₅ greater than 0.42.

9. The process of claim 8, wherein in (a) the organic solvent comprises ethyl acetate.

10. The process of claim 8, wherein in (b) precipitation is assisted by cooling below about 10° C.

11. The process of claim 8, wherein in (d) a powder is formed having ratios of D₅₀ to D₉₅ greater than about 0.43.

12. The process of claim 8, wherein in (d) a powder is formed having ratios of D₅₀ to D₉₅ greater than about 0.44.

13. The process of claim 8, wherein in (d) a powder is formed having ratios of D₅₀ to D₉₅ greater than about 0.45.

14. The process of claim 8, wherein in (d) ratios of D₅₀ to D₉₅ are not greater than 0.6.

15. The process of claim 8, wherein D₅₀ sizes are less than about 10 μm.

16. The process of claim 8, wherein D₅₀ sizes are about 3 μm to about 7 μm.

17. A solid pharmaceutical composition comprising an escitalopram oxalate powder of claim 1.

18. A solid pharmaceutical composition comprising an escitalopram oxalate powder of claim 2.

19. A solid pharmaceutical composition comprising an escitalopram oxalate powder of claim 3.

20. A solid pharmaceutical composition comprising an escitalopram oxalate powder of claim 4.

21. A solid pharmaceutical composition comprising an escitalopram oxalate powder of claim 5.
